(19) United States
(12) Patent Application Publication
(10) Pub. No.: US 2016/0000652 A1
(43) Pub. Date: Jan. 7, 2016

(54) CONTAINER AND KIT FOR PROVIDING PARENTERAL NUTRITION

(71) Applicant: EUROZYTO GMBH, Königstein (DE)

(72) Inventor: Uwe-Bernd Rose, Koenigstein (DE)

(21) Appl. No.: 14/770,337

(22) PCT Filed: Oct. 22, 2014
(86) PCT No.: PCT/EP2014/072646

§ 371 Date: Aug. 25, 2015

(30) Foreign Application Priority Data
Feb. 7, 2014 (DE) 102014202261.4

(51) Int. Cl. A61J 1/20; A61M 5/162

(52) U.S. Cl. CPC A61J 1/2093; A61J 1/2034; A61J 1/2048; A61M 5/162

(57) ABSTRACT

The invention relates to a container (26), preferably a bag (26), for providing parenteral nutrition, said bag having an interior that is sub-divided into separate compartments (1, 2, 3, 4, 5, 6, 7, 8, 9) and said compartments (1, 2, 3, 4, 5, 6, 7, 8, 9) being used to hold liquids for parenteral feeding. The container is designed and further developed with regard to administering parenteral nutrition according to the individual metabolism of a patient and with regard to simplicity of use, such that at least two compartments (1, 2, 3, 4, 5, 6, 7, 8, 9) are formed in the interior and a liquid connection can be produced between said compartments. Also disclosed is a kit for providing parenteral nutrition.

### CROSS REFERENCE TO RELATED APPLICATIONS

This application is a national stage application, filed under 35 U.S.C. § 371, of International Application No. PCT/EP2014/072646, filed Oct. 22, 2014, which claims priority to German Application No. 10 2014 202 261.4, filed Feb. 7, 2014, the contents of both of which as are hereby incorporated by reference in their entirety.

### BACKGROUND

1. Technical Field

The invention relates to a container, preferably a pouch, for the provision of parenteral nutrition, with an inner compartment subdivided into separate chambers, wherein the chambers serve the purpose of accommodating liquid parenteral nutrition.

The field of medicine distinguishes between enteral and parenteral nutrition. Parenteral nutrition is a form of artificial feeding which, due to an acute illness of the stomach and/or intestines, circumvents the gastrointestinal tract. Nutrition is generally given in this case using special solutions which are infused into one of the large veins of the body intravenously. For this reason, the solutions used for parenteral nutrition differ in their compositions of enteral nutrient solutions.

Parenteral solutions can be administered in different ways.

A first possibility is that of providing a pre-manufactured multi-chamber pouch in which the nutrition components are filled separately into different chambers. The components are mixed together as a result of the chamber walls being dissolved or destroyed shortly before the application. Such ready-to-use pouches have a pharmaceutical approval and always have the same composition. As a result, it is not possible to sufficiently take into account the individual metabolic situation of a patient. In addition, needed food components or drugs must be injected individually.

As an alternative, the patient can receive a customized nutrition solution corresponding to his metabolic status at a pharmacy or from a production facility approved for this purpose according to the AMG (Arzneimittelgesetz—German Drugs Acts) § 13. Sterile containers are available for this purpose, having a maximum of three chambers. The shelf life of the nutrition solution is reduced to a few days for single-chamber pouches. They must also be kept cool, which results in the manipulation by care providers and patients being difficult and critical to safety. The problem which exists for pre-manufactured pouches, that additionally needed nutrition components or drugs must be injected separately, also occurs for multi-chamber pouches with three chambers.

2. Description of Related Art

Such multi-chamber pouches are known from EP 0 619 998 B1, EP 0 893 982 B1 and EP 0 883 396 B1, for example.

### BRIEF SUMMARY

The problem addressed by the present invention is therefore that of designing and implementing a container, as well as a set, of the type indicated above, in such a manner that it is possible to administer parenteral nutrition according to the individual metabolic status of a patient, with easy handling.

According to the invention, the problem above is addressed by a container having the features of claim 1. According to the same, the container in question is designed and implemented in such a manner that at least two chambers are constructed in the inner compartment, wherein it is possible to produce a fluid connection between the two chambers.

It has been recognized according to the invention that the handling during a parenteral feeding is significantly simplified if multiple nutrition components, such as a parenteral nutrition solution and a liquid used to cover fluid requirements, can be provided at the same time. In this way, it is easy to cover a basic requirement for nutrition.

The container of the type indicated above is designed and implemented in such a manner that it is possible to administer parenteral nutrition according to the individual metabolic status of a patient, with easy handling.

The fluid connection between the two chambers can preferably be regulated, wherein such a controllability should be understood as an embodiment of a container according to the invention.

As such, it has been recognized that there is a special advantage to a configuration wherein, rather than the liquid components always being mixed in the same way, it is possible to provide an individual mixing according to the actual needs of a patient by regulating the fluid connection between the chambers. The fluid connection can be regulated in this case by opening and closing the fluid connection, for example a channel constructed between the chambers, or by partially opening or partially closing the same. In other words, the degree of opening of the fluid connection of the channels which implement the fluid connection can be regulated. It is possible to re-fill a chamber due to the fact that a chamber can be closed by the regulation after opening. It is also possible to administer nutrition components intermittently, for example using a fluid connection which only occurs intermittently.

As far as the concrete design of the container is concerned, it can be contemplated that the same is constructed as a pouch. Specifically, this pouch can be constructed as a polymer multi-chamber pouch. The pouch can consist of a flexible plastic film in two layers, particularly “turned back” on one side. The open sides can be closed by welding. A pouch with a length of 600 mm and a width of 250 mm has been demonstrated to be an advantageous size with sufficient volume. Due to the flexibility of the pouch made of plastic, the same can also be easily used for portable applications where the patients value mobility and the pouch is emptied by a mobile pump.

In one advantageous embodiment, at least one first chamber contains a parenteral nutrition solution with a first amount of energy, and at least one second chamber contains a liquid for the purpose of covering fluid requirements, with a second amount of energy, which is smaller than the first amount of energy. As such, the container is ready for use and the parenteral feeding can proceed. Specifically, the first chamber can have a greater volume than the second chamber. As such, a greater volume of the parenteral nutrition solution is provided. This configuration realizes an adequate adaptation of the parenteral nutrition, such that it covers needs for energy and nutrients. Due to the fact that the parenteral nutrition solution already has a certain liquid content, it is possible for the volume of the second chamber, containing the liquid used to cover fluid requirements, to be accordingly smaller. In this case, it can be contemplated that the first chamber contains 750 to 1000 ml of parenteral nutrition solution, and/or that the second chamber contains 250 to 750 ml of liquid used to cover fluid requirements.

For the adaptation of the parenteral nutrition to the requirements of a patient to be as multi-faceted as possible, additional chambers can be included in the inner compartment, having different sizes—preferably in progressive stages—to provide liquids in volumes matching requirements. As such, it is possible to provision additional liquids, specifically according to the concrete or potential needs of a patient. Depending on the individual needs of the patient, chambers are emptied individually or together or are mixed with each other, particularly by means of the controllable fluid connection between the chambers. The additional chambers can contain additional energy-bearing, parenteral nutrition components or other vitamin or mineral components. This further improves the provision of nutrition which covers requirements. It is also a very significant advantage that the parenteral nutrition solution which [contains] liquid to cover the fluid requirements, as well as further nutrition components, are provided in such a manner that it is possible to match the individual requirements of the patients as regards the composition and the amount of the liquids.

By way of example, it is possible to administer only the parenteral nutrition system from the one chamber, or only the liquid from the other chamber to cover the need for fluid, or both of these; and optionally additional components can be administered as well, which can be mixed immediately prior to the administration by the controllable fluid connection. What is essential is that all liquids are provided in the container. A very significant advantage for handling is that the container can be applied by the patient himself, because there is nothing else which needs to be additionally gathered or injected. Only a simple training of the patient is necessary for this purpose. Due to the resulting freedom from the need for a care provider, the patient is flexible and the treatment of the patient is more cost-effective.

Specifically, the inner compartment of the container can be subdivided into at least six, and preferably maximally ten—and more preferably nine—chambers. In this way, it is possible to provision different nutrition components by providing different fluids, such that an adequate parenteral nutrition is thereby possible. This number of chambers also enables the production of a sufficient number of “standard configurations” which are based on individualized formulations and—as a result of the components being stored in separate chambers—can be stored for longer. According to investigations, it is consequently possible to reproduce approximately 70 to 80% of the individualized formulations using approximately 15 to 20 standard configurations.

The liquid used to cover fluid requirements can have water and/or isotonic saline solution. The parenteral nutrition system can have a source of protein, carbohydrates, fats, vitamins, trace elements, electrolytes, and/or minerals. The additional, at least six, chambers can have fat emulsions, carbohydrate solutions, amino acid solutions, mineral solutions, trace element solutions, and/or vitamin solutions.

The parenteral nutrition system has, to cover the energy requirements of the patient, an amount of energy of at least 0.5 kcal/ml, preferably at least 0.8 kcal/ml, and particularly preferably at least 1 kcal/ml. Amino acids and the derivatives thereof can be used as the source of protein. Poly- and oligosaccharides can be used as the carbohydrates. Soybean oil, as well as fish oil, MCT (medium chain triglycerides), olive oil, omega-3 fatty acids, and structured triglycerides can be considered for the source of fat.

To produce the fluid connection, controllable channels can be included between the chambers, particularly channels which can open at least partially and which can close again at least partially. As such, the individual parenteral components can be mixed in a specific way, and thereby the parenteral nutrition can be prepared, using simple constructive measures. In addition, the nutrition components can be stored in a stable manner, because the liquids are only mixed together immediately prior to the administration of the parenteral nutrition. In one embodiment which includes reclosable channels, one component can be given a second time such that it is possible to administer a component according to needs, and by way of example intermittently.

Specifically, it can be possible to produce a fluid connection by means of the channels between additional chambers and the first chamber and/or the second chamber. The first and the second chamber in this case can serve as the discharge chambers. In addition, it is also possible to “premix” individual components, such that a mixing can take place in both the first chamber and the second chamber prior to the mixing of the liquids. As such, the first chamber and the second chamber can also be termed “prechambers”. In this context, it is advantageous if one or more of the additional chambers can be connected to the first chamber, and the remaining chambers of the additional, at least 6 others can be connected via their channels to the second chamber.

As part of an embodiment with an advantageous construction, the controllable channels can each be formed by a passage in the wall of one of the chambers and by a clamp means for the purpose of opening and closing the passage, particularly a clip. In addition to providing an advantageous construction, this configuration also offers easy handling, wherein the clamp means can be entirely or partially removed from the section of the container which forms the passage, thereby opening the channel entirely or partially. The degree of opening of the channel in this case can be directly seen, specifically by the presence, the absence or the position of the clamp means, particularly the clip. In one embodiment of the clamp means as the clip, a passage can be re-closed in a simple manner by the clip being “stuck back” on. In this case it can be contemplated that one clamp means or one clip is used for each passage. If multiple nutrition components can only be used together, it can also be contemplated that only one clip is functionally assigned to multiple passages.

To realize safe handling, an opening can be included for receiving and/or inserting the clamp means, particularly a clip, said opening projecting through the inner compartment and being separate from the same. The clamp means or the clip can be inserted through the opening and therefore attached to the passage. A corresponding removal thereof can also be realized in this manner. This design enables a secure alignment of the clamp means on the container, because the same do not protrude outward, which very significantly reduces the risk of an accidental removal of the clamp means or clip.

For rapid and flexible usability, a transfer system which can be coupled to one of the chambers can be included for the administration to a patient of the parenteral nutrition system and/or the liquid used to cover fluid requirements. Infusion systems can be used as the transfer system, having a tube and corresponding connectors. On one end can have [sic] a connector piece for the container and on the other end a connector piece for an intravenous application.

Specifically, a connector piece, particularly a Luer lock valve port, can be functionally assigned to at least the further chambers, for the purpose of filling or emptying the contents of the chamber. As such, individual chambers can be filled or emptied individually. In addition to a targeted administration of the contents of one chamber, a refilling of the same is therefore also possible.

To realize the most flexible possible usability, the first chamber and/or the second chamber can have at least one access, particularly a needle injection port with membrane, a Luer lock injection port, a connector for an infusion device with a Luer lock connection, and/or a spike port for an infusion device. In addition to flexible usability, the configuration therefore also offers high compatibility in the application of the container. In addition, the configuration realizes redundancy in this manner in cases where one of the connectors is damaged at the site of use, or the complementary counterpart thereof—for example connected to the pump—is not even available. With regards to the concrete arrangement of the ports and connectors listed above, it can be contemplated that the same are constructed on one side of the container.

With regards to a possible administration by means of gravity, suspension points, particularly in the form of perforations, can be included on the side opposite the port(s), for the purpose of attaching the container. As such, the configuration then ensures the administration of parenteral nutrition even if there are no complex medical devices available, such as dose pumps. Due to the construction of suspension points, the container can be attached at a point which is elevated over the patient. In this case, as mentioned above, it is advantageous if the connectors or discharge points of the first and/or the second chamber are arranged on one side of the container which is opposite the suspension points. To ensure gravity discharge of liquids from each of the chambers, it can be contemplated—in the case of a substantially rectangular design of the container—that there are perforations at each corner to form holding points.

The problem named above is also addressed by a set having the features of claim 10. For the set according to the invention, for the provision of parenteral nutrition, it is particularly important that the same has a container according to the invention and a transfer system which can be coupled to one chamber of the container for the purpose of administering parenteral nutrition to a patient, wherein the container and the transfer system are accommodated in a closed packaging.

This set enables the direct application of the parenteral nutrition system and/or the liquid used to cover fluid requirements, wherein the container, particularly the pouch, is contained in the packaging along with the transfer system or spiral tube. It is advantageous in this case if the container and the transfer system are kept sterile in the packaging. For this purpose, the packaging, which is preferably designed as a pouch, can consist of a film which makes possible a sterilization of the container, particularly the multi-chamber pouch. In addition, it is advantageous for the immediate applicability of the set if at least the parenteral nutrition system and the liquid used to cover fluid requirements are already contained in the container, such that the container and therefore the set as a whole are ready to use.

Infusion systems can be used as the transfer system or spiral tube, having a tube with corresponding connector pieces. A connector piece for the container can be included on one end, and the other end can have a connector piece for an intravenous application. The packaging can be designed as a film pouch to enable the simplest possible removal of the container and the transfer system from the packaging. The film pouch can be constructed of a flexible plastic film which is folded on one side, and is closed on the other side by a welded seam. Simple removal of the container and the transfer system is possible due to the design of an opening region, particularly without contaminating these components to the greatest degree possible. The region which can be opened can be designed as a separable seam, particularly a separable seam section of the welded seam, or as a perforation which enables the easy manual opening of the packaging.

### BRIEF DESCRIPTION OF THE FIGURES

At this point, there are various options for embodying and implementing the teaching of the present invention in an advantageous manner. For this purpose, reference is hereby made on the one hand to the claims, and on the other hand to the following explanation of preferred embodiments of the invention with reference to the drawings. Along with the explanation of the preferred embodiments of the invention and of the drawings, other generally preferred embodiments and implementations of the teaching are explained as well. In the drawings:

FIG. 1 shows a schematic view of a first embodiment of a container according to the invention, for the provision of parenteral nutrition,

FIG. 2 show a schematic view of one embodiment of a set according to the invention for the provision of parenteral nutrition, having a container according to the invention, and

FIG. 3 shows a schematic view of a second embodiment of a container according to the invention.

### DETAILED DESRIPTION OF VARIOUS EMBODIMENTS

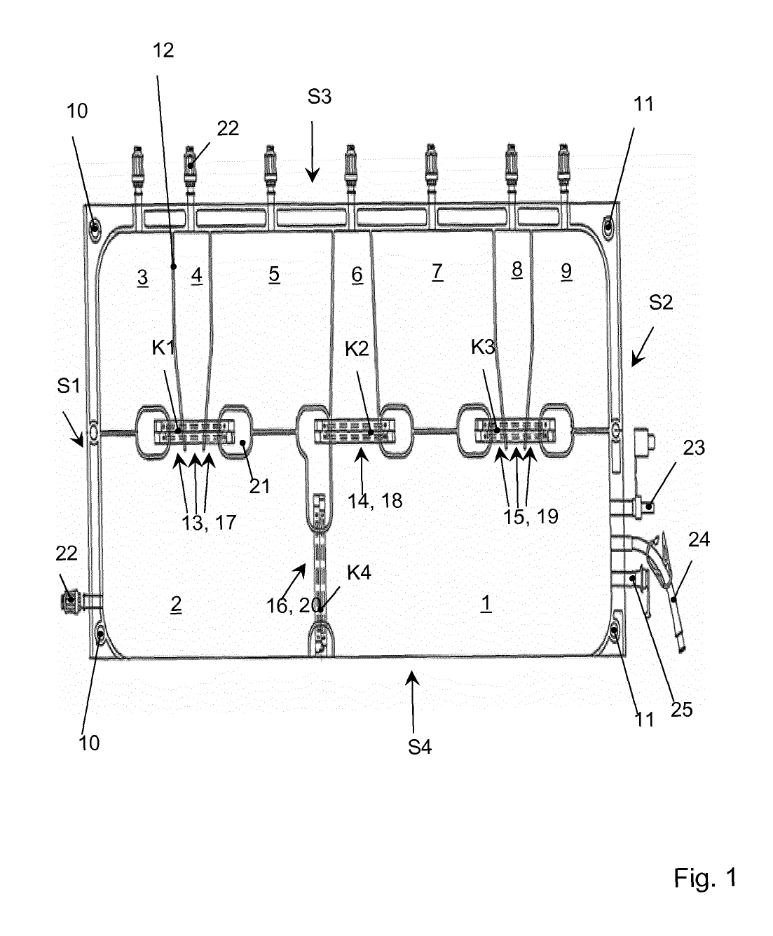

FIG. 1 shows one embodiment of a container according to the invention, for the provision of parenteral nutrition. The container is designed as a pouch, and particularly as a polymer multichamber pouch. The multichamber pouch has nine chambers 1 to 9 which are separated from each other.

The pouch is made of a flexible plastic film which is folded back, closed to the outside on the first side 51 and the second side S2 with a double welded seam, and on the third side S3—above in FIG. 1—with a single welded seam. The fourth side S4 does not need a welded seam, because the film is folded back at this point and therefore is closed without a seam.

Attachment points 10 in the form of perforations 10 are constructed on the first side 51 for the attachment of the pouch. Attachment points 11 in the form of perforations 11 are constructed on the second side 2. As such, liquid can be emptied by the action of gravity, wherein the pouch must be suspended in such a manner that the discharge point faces the floor, or—more simply put—“downward”.

The chambers 1 to 9 are separated from each other by solid chamber walls 12—only indicated once with reference numbers to simplify the depiction. The chambers 3, 4, and 5 each have a controllable channel 13 which can be used to produce a fluid connection to the second chamber 2. The chamber 6 has a channel 14, and the chambers 7, 8, and 9 each have a controllable channel 15. As such, the chambers 6, 7, 8, and 9 can be connected to the first chamber 1 by a fluid connection.

The first chamber 1 and the second chamber 2 can be coupled to each other by a channel 16 for the purpose of producing a fluid connection. The controllable channels 13, 14, 15, 16 are each formed by a passage 17, 18, 19, 20, and by clamp means K1, K2, K3, K4 for the purpose of opening and closing the passage 17, 18, 19, 20. Partial opening or partial closing is also possible. The clamp means K1, K2, K3, K4 are designed as clips K1, K2, K3, K4. The clamp means or clips K1, K2, K3, K4 can be removed entirely or partially such that the fluid volume which can be removed from a chamber can be regulated. The reattachment of the clamp means or clips K1, K2, K3, K4 can also be contemplated, such that it is also possible to reclose and optionally refill a chamber.

Openings 21—only indicated once with reference numbers, to simplify the depiction—which project through the inner compartment of the pouch and are separate from the same, are included for the purpose of receiving or inserting the clips K1, K2, K3, K4. In this way, the clips K1, K2, K3, K4 are arranged in the interior of the pouch and do not protrude from the side of the pouch, such that the risk of an accidental removal of the clips is minimized. At the same time, it is possible to directly recognize, by the presence, absence, or the position of the clips K1, K2, K3, K4, whether the passages 17, 18, 19, 20 are open or closed, or are partially open or closed. The clamp means or clips K1, K2, K3, K4 can also be opened individually, such that it is possible to realize individualized mixtures according to the respective liquids in the chambers 1 to 9. In this way, it is possible to address the individual metabolic status of a patient.

Each of the chambers 2 to 9 has a connector piece 22 assigned to it for the purpose of filling or emptying the contents of the chamber. The connector piece 22 is designed in the present embodiment as a Luer lock valve port 22. As such, not only is a targeted removal or application of one of the liquids possible, but also a needed liquid can be filled or refilled.

The first chamber 1 has a needle injection port with membrane 23, a Luer lock injection port or a connector for an infusion device with a Luer lock connection 24, and a port for an infusion device with a spike 25. This configuration enables a flexible connection possibility for the pouch 1, for the provision of parenteral nutrition. As such, the configuration also realizes a redundant connection possibility if the complementary counterpiece to one of the connectors constructed on the pouch is defective or is not even available at the use site.

The channels 14 and 16 each have one clip K2, K4. The channels 13 and 15 of the chambers 3, 4, 5 and 7, 8, 9 each have one clip K1, K3 for multiple channels.

In the present embodiment, the pouch has a length of 600 mm and a width of 250 mm. The chambers 1 to 9 have different sizes, specifically with a range of stages. As such, it is possible to store different liquids in the pouch in the amounts needed. In addition, the first chamber 1, which serves the purpose of holding the parenteral nutrition system, has a greater volume than the second chamber 2, which serves the purpose of holding a liquid used to cover fluid requirements. The first chamber 1 has a capacity of 1000 ml, while the second chamber 2 can have a volume of 600 ml. The chambers 5 and 7 each have a capacity of 100 ml, whereas the chambers 4, 6, and 8 each have a capacity of 15 ml. The chambers 3 and 9 can each have a volume of 80 ml.

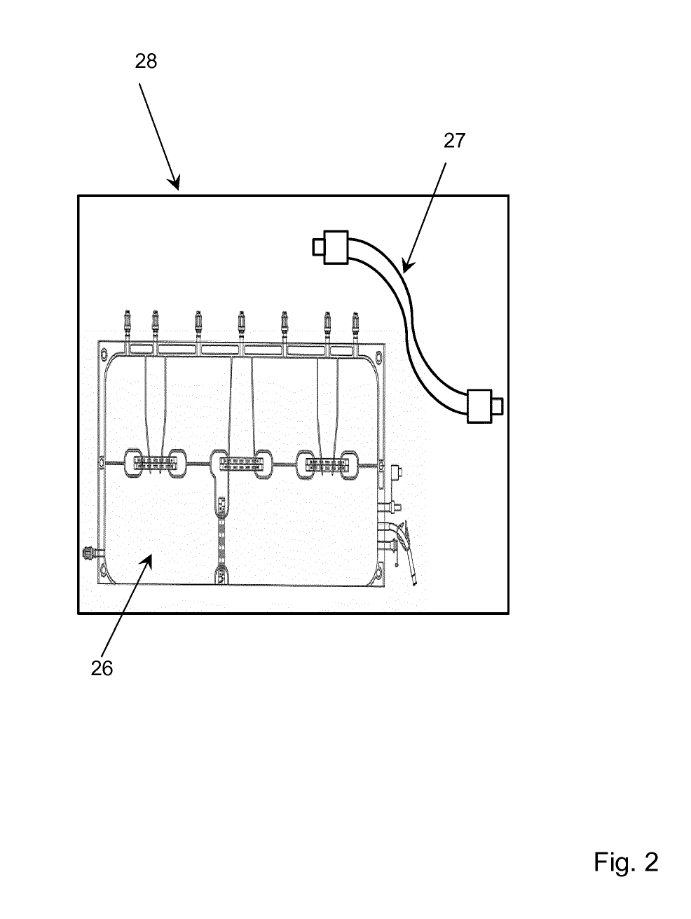

FIG. 2 shows one embodiment of a container according to the invention, for the provision of parenteral nutrition. The set includes a container 26 according to the invention with parenteral liquids contained therein, particularly at least one parenteral nutrition system and one liquid used to cover fluid requirements. In addition, the set also contains a transfer system 27 for the administration of parenteral nutrition to a patient, which can be coupled to one of the chambers of the container 26 and which is only illustrated schematically in this case. An infusion system having a tube and corresponding connectors can be used as the transfer system. A connector piece for connection to the container is constructed on one end, and a connector piece for an intravenous application is constructed on the other end.

The container 26 and the transfer system 27 are held in a closed and sterile packaging 28. The packaging 28 is made as a pouch of superimposed plastic films which are connected to each other by a peripheral welded seam, which is not illustrated here. At least a part of the welded seam—which is not illustrated—is constructed as a tear-open or separable welded seam, which can be called a peel seam. Therefore, it is possible to open the sterile packaging 28, to remove the components of the set, in a simple manner. As a result of the fact that the set provides the pouch 26 and the transfer system 27 in the packaging 28 in a sterile and ready-to-use state, the parenteral nutrition can be administered immediately and quickly, because the packaging 28 need only be opened, after which the parenteral nutrition can be applied.

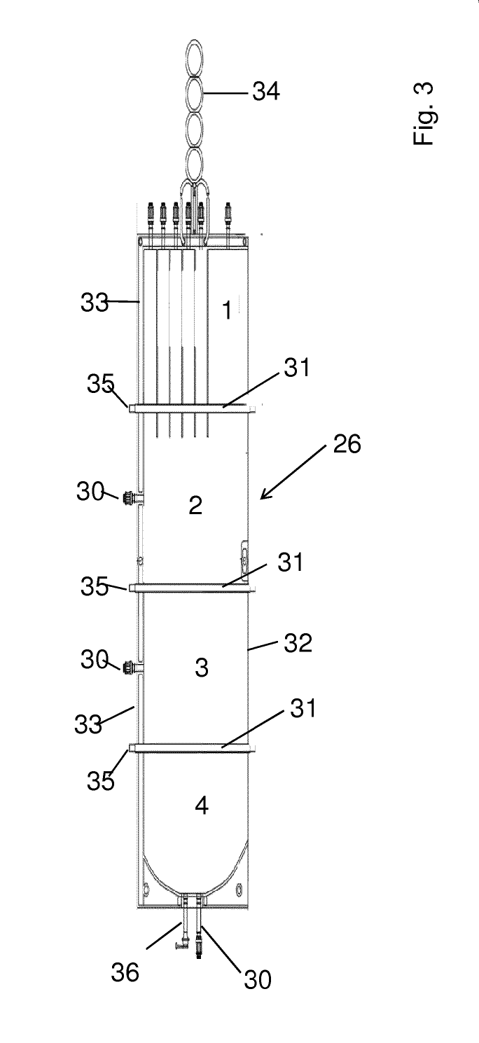

FIG. 3 shows a further embodiment of a container (26) according to the invention, wherein the container (26) consists of a folded and welded film sheet (32). A type of frame (33) is included to hold and/or receive the pouch (26), serving as a receptacle and/or suspension for the container (26). The frame (33) also has a suspension device (34) which can be formed by an arrangement of ring eyelets or the like.

For the container (26) shown in FIG. 3, it is essential that the same is subdivided by a total of three clamping rails (31) into four chambers 1, 2, 3, 4, wherein the clamping rails have an inserted and/or integrated clamp band (35). If the clamp band (35) is fully or partially pulled out onto the clamping rail (31), a fluid connection is produced between the chambers 1, 2, 3, 4 and the fluids in the chambers 1, 2, 3, 4 in the container (26) can be specifically mixed.

In the embodiment illustrated in FIG. 3, there is no possibility of regulating a fluid connection between the chambers 1, 2, 3, 4, such that the fluids mix entirely following the removal of the clamping band (35). If one wishes to use the contents of a chamber partially or not at all, the chamber can be partially or entirely emptied via a valve (30).

After the contents of the chambers 1, 2, 3, 4 have been mixed, the mixture can be administered via the outlet valve (36).

As regards further advantageous embodiments of the container according to the invention, as well as the set according to the invention, reference is hereby made for the purpose of avoiding repetition to the general portion of the description and to the attached claims.

Finally, explicit reference is made to the fact that the embodiments of the container according to the invention, and the set according to the invention, described above, merely serve to explain the claimed teaching, but the teaching is not restricted to the embodiments.

### LIST OF REFERENCE NUMBERS

1 first chamber
2 second chamber
3, 4, 5, 6, 7, 8, 9 chambers
10, 11 suspension point, perforation
12 wall
13, 14, 15, 16 channel
17, 18, 19, 20 passage
21 opening
22 connector piece, Luer lock valve port
23 needle injection port
24 Luer lock injection port
25 port for the spike-port infusion device
26 container, pouch, polymer multichamber pouch
27 transfer system
28 packaging
30 valve
31 clamping rail
32 film sheet
33 frame/subframe
34 suspension device
35 clamp band
36 outlet valve
K1, K2, K3, K4 clamp means, clip
S1, S2, S3, S4 side

1-14. (canceled)

15. A container (26) for the provision of parenteral nutrition, the container comprising an inner compartment subdivided into separate chambers (1, 2, 3, 4, 5, 6, 7, 8, 9), wherein the chambers (1, 2, 3, 4, 5, 6, 7, 8, 9) serve the purpose of accommodating liquids for parenteral nutrition, wherein at least two chambers (1, 2, 3, 4, 5, 6, 7, 8, 9) are constructed in the inner compartment, and wherein it is possible to produce a fluid connection between the at least two chambers (1, 2, 3, 4, 5, 6, 7, 8, 9).

16. A container according to claim 15, wherein the fluid connection between the chambers (1, 2, 3, 4, 5, 6, 7, 8, 9) can be regulated.

17. A container according to claim 15, wherein the chambers (1, 2, 3, 4, 5, 6, 7, 8, 9) are configured to be opened outwardly via a valve (30), and to be emptied to the outside at least one of entirely, partially, or in a dosed manner.

18. A container according to claim 15, wherein:
at least one first chamber (1) contains a parenteral nutrition system with a first amount of energy;
at least one second chamber (2) contains a liquid used to cover fluid requirements, with a second amount of energy which is less than the first amount of energy of the parenteral nutrition system; and
the first chamber (1) has a greater volume than the second chamber (2).

19. A container according to claim 15, wherein there are additional chambers (3, 4, 5, 6, 7, 8, 9) in the inner compartment which have different sizes, particularly in progressive stages, for the provision of liquids in volumes which match needs

[symbol]

20. A container according to claim 15, wherein the inner compartment is subdivided into at least six and no more than a maximum of ten chambers.

21. A container according to claim 15, wherein the inner compartment is subdivided into nine chambers (1, 2, 3, 4, 5, 6, 7, 8, 9).

22. A container according to claim 15, wherein the chambers are formed and/or delimited from each other by clamping rails (31) which extend transverse to the longitudinal direction of the pouch (26).

23. A container according to claim 22, wherein:
bands are arranged in the clamping rail, which clamp into the clamping rails; and
a fluid connection is created between the chambers (1, 2, 3, 4, 5, 6, 7, 8, 9) when the bands are pulled out.

24. A container according to claim 15, wherein:
controllable channels (14, 15, 16, 20), which can be at least partially opened and at least partially reclosed, are included between the chambers (1, 2, 3, 4, 5, 6, 7, 8, 9) for the purpose of producing the fluid connection; and
a fluid connection between additional chambers (3, 4, 5, 6, 7, 8, 9) and the first chamber (1) and/or the second chamber (2) can be produced by means of the channels (13, 14, 15, 16).

25. A container according to claim 24, wherein the controllable channels (13, 14, 15, 16) are each formed by a passage (17, 18, 19, 20) in a wall (12) of one of the chambers (1, 2, 3, 4, 5, 6, 7, 8, 9) and by a clamp means (K1, K2, K3, K4) for the purpose of opening and closing the passage (17, 18, 19, 20), particularly a clip (K1, K2, K3, K4).

26. A container according to claim 25, wherein an opening (21) which projects through the inner compartment and is separated from the same is included for the purpose of receiving and/or inserting the clamp means (K1, K2, K3, K4), particularly the clip (K1, K2, K3, K4).

27. A container according to claim 15, wherein:
a transfer system (27) configured to be coupled to one of the chambers (1, 2, 3, 4, 5, 6, 7, 8, 9) is included for the purpose of administering the parenteral nutrition system and/or the liquid used to cover fluid requirements to a patient; and
at least one connector piece (22) is functionally assigned to the additional chambers (3, 4, 5, 6, 7, 8, 9) for the purpose of filling or emptying the contents of the chamber—particularly a Luer lock valve port (22).

28. A container according to claim 15, wherein at least one of the first chamber (1) or the second chamber (2) have at least one access (22, 23), particularly a needle injection port (23) with membrane, a Luer lock injection port (24), a connector for an infusion device with a Luer lock connection, and/or a spike port (25) for an infusion device.

29. A container according to claim 28, wherein suspension points (10, 11) for the attachment of the container, particularly in the form of perforations (10, 11), are included on the side which is opposite the access (22, 23, 24, 25).

30. A set for the provision of parenteral nutrition, said set having a container (26) according to claim 15 and a transfer system (27) configured to be coupled to a chamber of the container (26), for the administration of parenteral nutrition to a patient, wherein the container (26) and the transfer system (27) are held in a closed packaging (28).
